(19) United States
(12) Patent Application Publication
(10) Pub. No.: US 2022/0079848 A1
(43) Pub. Date: Mar. 17, 2022

(54) 4-in-1 Facial and Body Wipe

(71) Applicant: Tricia Morris, Highland Beach, FL (US)

(72) Inventor: Tricia Morris, Highland Beach, FL (US)

(21) Appl. No.: 17/447,785

(22) Filed: Sep. 15, 2021

Related U.S. Application Data
(60) Provisional application No. 62/706,908, filed on Sep. 17, 2020.

(51) Int. Cl. A61K 8/02; A61K 8/34; A61K 8/49; A61K 8/64; A61K 8/9794; A61K 8/27; A61K 8/37; A61Q 19/10; A61K 8/60; A61Q 19/00; A61K 8/365; A61K 8/43; A61K 8/9789

(52) U.S. Cl. CPC A61K 8/0208; A61K 8/9789; A61K 8/34; A61K 8/498; A61K 8/4913; A61K 8/64; A61K 8/9794; A61K 8/27; A61K 8/37; A61Q 19/10; A61K 8/604; A61Q 19/007; A61K 8/365; A61K 8/43; A61K 8/345

(57) ABSTRACT

The present invention relates to a combination of ingredients wherein it is possible to provide a solution or composition that provides all the advantages of skin cleansing, skin moisturizing, skin repair and skin protection. The present invention relates to facial and/or body wipe that incorporates a combination of ingredients such that a single wipe provides for all of skin cleansing, skin moisturizing, skin repair and skin protection. The present invention relates to an improved facial and/or body wipe that provides for improved advantages with respect to all of skin cleansing, skin moisturizing, skin repair and skin protection.

### CROSS-REFERENCE TO RELATED APPLICATIONS

The current application claims priority to U.S. Provisional Patent Application Ser. No. 62/706,908, filed on Sep. 17, 2020, which is herein incorporated by reference in its entirety.

### FIELD OF THE INVENTION

The present invention relates generally to facial and body wipes. The present invention relates to a facial and body wipes that dean, moisturize, repair and protect the skin.

### BACKGROUND OF THE INVENTION

Aging of the skin is a complex process in which damage of the skin from environmental factors plays an important role. Maintenance of the skin to minimize such damage and minimize at least the visual effects of aging is an even more complex process that may involve multiple components. Important components of such skin maintenance include at the very least routines for cleaning, moisturizing, healing and protecting the skin. The use of facial and body wipes has become a routine part of many such skin maintenance routines.

However, traditional wipes available in the marketplace are simple 2-in-1 wipes, namely a single wipe that provides for only cleansing and moisturizing of the skin. Moreover, traditional wipes for skin resurfacing or exfoliating such as skin peels contain strong ingredients that are harsh on the skin. Such traditional wipes do not provide for any healing or protection for the skin. Moreover, traditional wipes contain alcohol which both strips and dehydrates the skin. Traditional wipes may also leave a sticky residue due to the cleansing agents contained therein. As such, when using a traditional wipe, consumers are forced to use additional products to also repair, heal and/or protect the skin from further damage, as well as to clean the skin from residues left on the skin by the traditional wipe.

Accordingly, there is a need for improved methods of skin maintenance including improved facial and body wipes for cleaning, moisturizing, repairing and protecting the skin.

### SUMMARY OF THE INVENTION

In an exemplary embodiment, the present invention provides for all-in-one facial and/or body wipes having a combination of ingredients so as to provide for cleaning, moisturizing, repairing and protecting of the skin. In an exemplary embodiment, the present invention provides for a woven or non-woven facial or body wipe that incorporates a solution or composition that provides for the cleaning, moisturizing, repairing and protection of the skin.

### BRIEF DESCRIPTION OF THE DRAWINGS

FIG. 1 is perspective view of a wipe and packaging therefore according to an embodiment of the present invention.

### DESCRIPTION OF THE INVENTION

The inventors have now discovered that it is possible to provide the advantages of skin cleansing, moisturizing, repair and protection in a single invention. The inventors have now discovered that through a combination of ingredients, it is possible to provide a solution or composition that provides all of the advantages of skin cleansing, moisturizing, repair and protection. The inventors have now discovered that it is possible to create a facial and/or body wipe that incorporates a combination of ingredients, such that a single wipe provides for all the advantages of skin cleansing, moisturizing, repair and protection.

Skin aging is a complex process in which time and environmental factors play important role. As such, skin care is an equally complex process in which several factors play a. role. To begin, proper skin care requires adequate cleaning of the skin. How a person washes their skin can make a significant difference in their appearance. That is, cleansing of the skin is extremely important because individuals must remove the build-up of dirt, oil and debris. By cleansing the face and body, pollutants, bacteria, viruses and dead skin cells are removed thereby allowing the epidermis layers to breathe and generate new skin cells. Excessive dirt causes the follicles to trap bacteria and harmful toxins under the skin causing inflammation and acne. Cleansing also helps maintain the pH levels of the skin which helps to prevent dehydration. Dehydrated skin will create rough skin resulting in deep wrinkles. By cleansing the skin, other ingredient that are important for the rejuvenation of the skin are allowed to penetrate the dermis layers resulting in clear skin free of irritation. Unfortunately, traditional wipes are inadequate for cleaning the skin. Traditional wipes contain chemicals such as 2-bromo-2-nitropropane-1,3-diol which not only fail to remove bacteria and build-up, but can damage the skin and thus such chemicals must be rinsed off. Traditional wipes may also include formaldehyde-releasing chemicals that are a form of carcinogen (or a cancer-causing agent). Other traditional wipes contain alcohol. Alcohol breaks down the skin's protective barrier, dries the skin out, and removes those substances (e.g., lipids, enzymes, and antioxidants) that keep the skin healthy. As such, alcohol results in skin being irritated, dry and open to infections. As such, the present invention may provide for means by which the skin is cleaned. To this end, the present invention may include a cleansing agent for the skin.

Proper skin care also requires moisturizing of the skin. Moisturizing of the skin is important because all skin types (oily, dry, sensitive and combination skin) should be moisturized daily. If skin is not moisturized, the pores of the skin remain open to environmental pollutants. By moisturizing, the pores are filled with those ingredients that are healthy to the skin and protect the skin from daily pollutants in the air. As such, moisturizing of the skin helps to prevent acne, the skin drying out, inflammation and wrinkles. Traditionally the skin is moisturized by applying moisturizing ingredients such as glycerin, biotin, pyrrolidone carboxylic acid (e.g., the sodium salt form of pyrrolidone carboxylic acid). As such, the present invention may provide for means by which the skin is moisturized. To this end, the present invention may include a moisturizing agent for the skin.

Proper skin care also requires supplementing the skin's natural recuperative and repair functions. While the skin naturally repairs itself when injured, the repair process can be improved by the daily application of specific reparative ingredients to injured skin. For example, the skin may be repaired by combining specific combinations of ingredients, as in on exemplary embodiment, the chrysin flower, glycolic acid, lactic acid, citric acid phytic acid, peptides, synthetic peptides, a synthetic compound peptide with several chains of amino acids, vitamin B3, marshmallow root extract, cucumber extract, and ash tree bark extract. Notably, peptides are amino acids that make up certain proteins the skin requires. For example, collagen is made of three polypeptide chains, so preferably adding peptides can stimulate your skin to make collagen. More collagen can lead to firmer and younger looking skin. Such compositions can improve skin repair. As such, the present invention may provide for means by which the skin is repaired. To this end, the present invention may include a repair agent for the skin.

Such ingredients of the invention are able to work synergistically while also providing the benefits of each ingredient separately. As exemplary benefits of such ingredients, the chrysin flower is an anti-anti-inflammatory that soothes and conditions skin. Glycolic acid is an exfoliator that removes dead cells. Lactic acid evens out the skin complexion, reduces pore size, and lightens sun spots and age spots. Citric acid, found in fruits, promotes skin re-growth. Phytic acid is an antioxidant that helps to prevent blackheads. Moreover, notable is palmitoyl tripeptide-1 with three amino acids (the building blocks of all proteins) peptide with the amino sequence of glycine-histidine-lysine, or GHK. GHK is attached to palmitic acid (a fatty acid) to increase oil solubility and skin penetration. Combining this (e.g., GHK) with the palmitoyl-7 stimulates collagen production resulting in fewer wrinkles. Furthermore, glycolic acid is effective for removing blackheads, whiteheads and pimples from skin. This benefit also helps to reduce pore size. Consistent and repeated use of glycolic acid peels has been shown to be effective for removing cystic lesions and acne scars from skin. The chamomile flower is a powerful anti-inflammatory that heals eczema and dermatitis. Marshmallow root extract relieves itching, swelling and redness. Cucumber extract repairs dry and damaged skin.

Proper skin care also requires that the skin be protected. While the skin is designed to protect itself from the environment, the skin is under constant attack from environment factors such as UVA, UVB, infra-red and visible light, cigarette smoke and/or pollution. Such environmental attacks either directly or indirectly result in skin cell damage, caused by the generation of reactive species, in particular reactive oxygen species, reactive carbonyl species and reactive nitrogen species. Such reactive species are harmful to skin, causing damage to biological macromolecules, oxidizing proteins, DNA and lipids. If the skin is left unprotected the resulting oxidative damage result in the increased appearance of lines and wrinkles, actinic lentigines, dyspigmentation, rough skin, loss of elasticity and firmness and sagging, all of which are generally known as signs of ageing. While the skin may be protected through the application of sunscreen as well as antioxidants, additional skin protection is required for adequate skin care. As such, the present invention may provide for means by which the skin is protected. To this end, the present invention may include a protection agent for the skin.

The present invention is able to provide a combination of ingredients to be able to accomplish all four tasks of cleaning, moisturizing, repairing and protecting in one facial or body wipe. Accordingly, the present invention provides users with a simple way to maintain their skin by providing facial or body wipes that can be easily stored and transported in a small bag or purse, for when the user engages in activities such as tennis, golf, yoga and dancing. In one exemplary embodiment, the present invention is suitable for any type of activity in which the user may perspire or be subjected to humidity which may compromise the pores of the skin, or be subjected to UVA and/or UVB rays. In one exemplary embodiment, the present invention is an all green, environmentally friendly biodegradable facial or body wipe made up of ingredients that cleanses (without stripping), moisturizes, repairs and protects the skin.

The present invention may include at least one first repair agent. Exemplary first repair agents of the present invention include glycerin, steareth-20, chrysin, N-hydroxysuccinimide, palmitoyl oligopeptide-1, palmitoyl tetrapeptide-7, niacinamide and/or combinations thereof. In a preferred embodiment, the first repair agent of the invention is Haloxyl®. By way of example, a first repair agent may be included in the present invention in an amount less than about 1%, preferably less than about 1% by weight. Additional first repair agents may also be included within the scope of exemplary embodiments of the present invention.

The present invention may include at least one second repair agent. Exemplary second repair agents of the present invention include propanediol, iris florentina root extract, zinc, retinyl palmitate, glycolic acid, citric acid, lactic acid, phytic acid and/or combinations thereof In a preferred embodiment, the second repair agent of the invention is cytobiol. By way of example, a second repair agent may be included in the present invention in an amount less than about 1%, preferably less than about 1% by weight. Additional second repair agents may also be included within the scope of exemplary embodiments of the present invention.

The present invention may include at least one third repair agent. Exemplary third repair agents of the present invention include vitamin B3, chamomile, oat, cucumber, marshmallow root, bark, ash tree bark extract and/or combinations thereof. In a preferred embodiment, the bark or bark extract is fraxinus excelsior bark extract. In a preferred embodiment, the repair agent is fraxinus excelsior bark extract. By way of example, a repair agent may be included in the present invention in an amount less than about 1%, preferably less than about 1% by weight. Additional repair agents may also be included within the scope of exemplary embodiments of the present invention.

The present invention may include at least one cleansing agent. Exemplary cleansing agents of the present invention include glycolic acid, lactic acid, steareth-20, caprylyl, capryl, glucoside, chlorhexidine glucoside, oat kernel extract, steareth-20, capryl, glucoside, chlorhexidine glucoside and/or combinations thereof. In a preferred embodiment, the cleansing agent is chlorhexidine gluconate, an antiseptic cleanser sometimes used before surgeries. By way of example, a cleansing agent may be included in the present invention in an amount more than about 1%, preferably more than about 1% by weight. Preferably, a cleansing agent may be included in the present invention in an amount between about 1% and 5%, preferably between about 1% and 5% by weight. Additional cleansing agents may also be included within the scope of exemplary embodiments of the present invention.

The present invention may include at least one moisturizing agent. Exemplary moisturizing agents of the present invention include glycerin, sodium PCA, biotin, & calcium gluconate and/or combinations thereof. In a preferred embodiment, the moisturizing agent is glycerin. By way of example, a moisturizing agent may be included in the present invention in an amount less than about 1%, preferably less than about 1% by weight. Additional moisturizing agents may also be included within the scope of exemplary embodiments of the present invention.

The present invention may include at least one protection agent. Exemplary protection agents of the present invention include aloe vera, apple and/or combinations thereof. In a preferred embodiment, the protection agent is aloe vera and/or apple. By way of example, a protection agent may be included in the present invention in an amount less than about 1%, preferably less than about 1% by weight. Additional protection agents may also be included within the scope of exemplary embodiments of the present invention.

The present invention may include at least one solvent and/or carrier. Exemplary solvents and/or carriers of the present invention include water, pentylene glycol and/or combinations thereof. In a preferred embodiment, the solvent and/or carrier is water. In another preferred embodiment, the solvent and/or carrier is pentylene glycol. By way of example, a solvent or carrier may be present in an amount more than about 1%, preferably more than about 1% by weight. Preferably, a solvent or carrier may be included in the present invention in an amount between about 1% and 5%, preferably between about 1% and 5% by weight. Additional solvents and/or carries may also be included within the scope of preferred embodiments of the present invention.

The present invention may include additional ingredients and combinations thereof. In one exemplary embodiment, the present invention may include a skin conditioner and/or anti-inflammatory agent. In another exemplary embodiment, the present invention may include a peptide, preferably peptides that may enhance the stability and skin penetration of the present invention, preferably peptides that may enhance the skin's appearance. In one preferred embodiment, the additional ingredient is chrysin. In another preferred embodiment, the additional ingredient is palmitoyl tripeptide-1 and/or palmitoyl tetrapeptide-7. in another preferred embodiment, the additional ingredient is palmitoyl tripeptide-1 and/or palmitoyl tetrapeptide-7, N-hydroxysuccinimide. In another preferred embodiment, the additional ingredient is chlorhexidine glucoside.

The present invention may include at least one solution or composition. In one exemplary embodiment, the solution or composition may include at least one repair agent. In another exemplary embodiment, the solution or composition may include at least one cleansing agent. In another exemplary embodiment, the solution or composition may include at least one moisturizing agent. In another exemplary embodiment, the solution or composition may include at least one protection agent. In another exemplary embodiment, the solution or composition may include at least one moisturizing agent, at least one cleansing agent, at least one moisturizing agent and/or at least one protection, and/or combinations thereof. In a further exemplary embodiment, the present invention may include additional ingredients or components that are suitable for use in the present invention.

The solutions or compositions of the invention may be formulated by those of ordinary skill in the art. Those of ordinary skill in the art will recognize that the solutions or composition may for formulated by standard formulation techniques known to those of ordinary skill in the art. In one exemplary embodiment, the solutions or composition may for formulated by the mixing of ingredients, the bulk mixing of ingredients and/or multiple phases of mixing ingredients as will be recognized by those of ordinary skill in the art.

In another exemplary embodiment, the present invention may include the following ingredients: N-hydroxysuccinimide, chrysin, palmitoyl tripeptide-1, palmitoyl tetrapeptide-7, butylene glycol, niacinamide, fraxinus excelsior bark extract, lactic acid and phytic acid.

In another exemplary embodiment, the present invention may include the following ingredients: water, sodium citrate, gluconolactone, pentylene glycol, lavandula angustifolia (lavender) oil, pyrus malus (apple) fruit extract, aloe barbadensis leaf extract, avena sativa (oat) kernel extract, chamomilla recututa (matricaria) flower extract, cucumis sativus (cucumber) fruit extract, althaea officinalis root extract, fraxinus excelsior bark extract, glycerin, sodium PCA, butylene glycol, niacinamide, silanetriol, caprylyl/capryl glucoside, sodium benzoate, steareth-20, tetrasodium EDTA, N-hydroxysuccinimide, chrysin, palmitoyl tripeptide-1, palmitoyl tetrapeptide-7, lactic acid and phytic acid.

In another exemplary embodiment, the present invention may include: glycolic acid, lactic acid, steareth-20, caprylyl/capryl and glucoside for skin cleansing; glycerin for skin moisturizing vitamin B3. chamomile, oat, cucumber, marshmallow root, bark, tetrapeptide-7, chrysin, haloxyl and cytobiol for skin repair, and aloe vera and apple for skin protection.

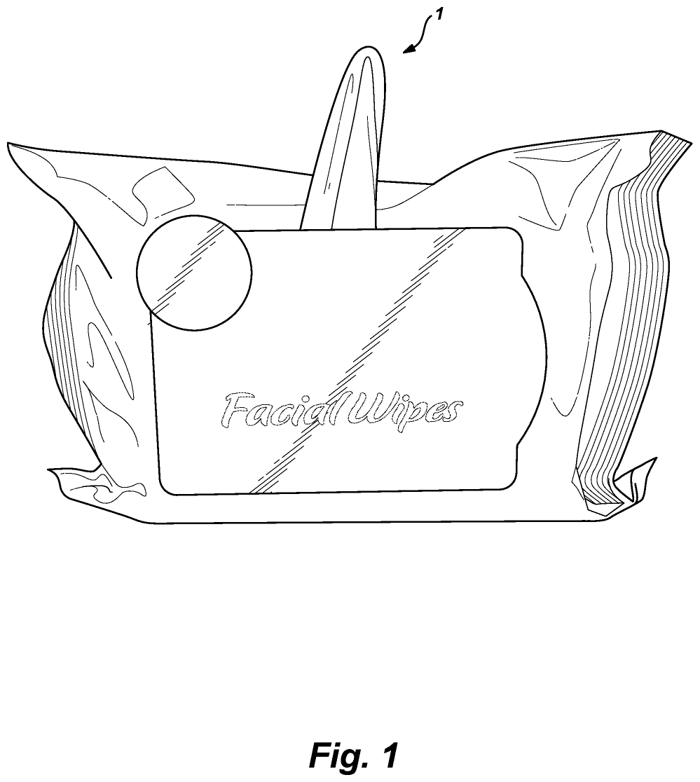

With reference to FIG. 1, the present invention may include a woven or non-woven fabric. In one exemplary embodiment of the present invention, the fabric is a facial wipe 1. In another exemplary embodiment of the present invention, the fabric is a body wipe 1. In another exemplary embodiment of the present invention, the fabric is a body and facial wipe 1. In an exemplary embodiment, the woven or non-woven fabric incorporates a solution or composition according to the present invention. The woven or non-woven fabric may be a facial or body wipe 1 product known to those of ordinary skill in the art. The invention may also include a package of multiple facial or body wipe 1 materials suitable for wiping a user's facial or body skin. The individual wipes 1 in the package may be in a stacked or layered arrangement, for example where the individual wipes 1 are stacked or layered one upon another.

The solutions or compositions of the invention may be incorporated onto or into the woven or non-woven fabrics by conventional techniques known to those of ordinary skill in the art. In one exemplary embodiment, the solutions or compositions of the present invention may be coated onto the fabrics as a continuous or discontinuous coating. Coating of the solutions or compositions of the invention can be carried out by conventional techniques known in the art including, as appropriate, solvent slot coating, dip coating, spray coating, roll coating, gravure coating, melt coating, transfer coating, or the like. In another exemplary embodiment, the solutions or compositions of the present invention may be incorporated into the fabrics by conventional techniques known in the art. Through laboratory trials, the solutions or compositions of the present invent incorporated into a wipe 1 have been tested to establish a year of stability testing to ensure the efficacy of the ingredients, proper pH levels and moisture sorption.

The present invention is able to provide for improved results not before seen. In the present invention the ingredients are able to work both independently as well as synergistically to achieve the benefits of skin cleansing, skin moisturizing, skin repair and skin protection. For example, glycolic acid is able to stimulate the skin to create more collagen, wherein collagen is the protein that gives skin its firmness, plumpness and elasticity. Lactic acid may be used to treat hyperpigmentation, age spots, and other factors that contribute to a dull and uneven skin complexion. Other benefits of AHAs like lactic acid are improved skin tone and reduced pore appearance. Glycerin promotes the synthesis of lipids in the epidermis, guiding cells to grow and mature properly, accelerating wound healing and reducing bruising. Glycerin has also been shown to help treat fungal infections in disorders like eczema and psoriasis. Cucumber is beneficial to those with irritated and acne-prone skin. For example, the soothing ingredients of cucumber water may be infused with bentonite clay to create a mask that can protect against skin irritation and swelling. Vitamin B3 is a water-soluble vitamin that works with the natural substances in the skin to help visibly minimize enlarged pores, tighten lax pores, improve uneven skin tone, soften fine lines and wrinkles, diminish dullness, and strengthen a weakened surface. Chamomile includes anti-inflammatory and antioxidant properties that can help soothe the skin, reducing redness and blemishes. It can even relieve skin irritations like eczema, psoriasis and rosacea. Marshmallow Root has healing, moisturizing and soothing properties that make it highly effective for revitalizing skin. Tetrapeptide-7 peptide is composed of four amino acids plus fatty acid palmitic acid to enhance stability and penetration into the skin and helps to replenish the skin's surface and calm visible signs of sensitization. Aloe reduces inflammation and protects the skin's barrier. Apple extract is rich in antioxidants that help protect against ultraviolet radiation and lead to younger looking skin. Apple extract is also rich in vitamin C, which prevents premature aging and promotes healthy, radiant skin. Cytobiol beneficially addresses the causes of the formation of the bags and dark circles. Ash bark extract brings fraxin & esculin which present a veinotonic, a “vitamin P activity” and anti-oxidant properties. Organic silicon has vasculo-protective activity to reinforce capillary structure. Vitamin B3 helps to restore normal blood flow. Haloxyl contains a combination of active peptides which brighten and thicken the skin. Those of ordinary skill in the art know that the skin around the eyes is naturally thinner and more susceptible to showing signs of ageing while also being one of the most difficult areas to treat. As such, this ingredient is able to brighten and fade away dark circles. Chrysin is a naturally occurring isoflavon that is extracted from plants, most often from the passiflora caeruiea (blue passion flower). Chrysin acts as a skin conditioner and anti-inflammatory agent while also demonstrating the ability to scavenge free radicals in the body thereby functioning as an antioxidant. All of such ingredients may be used in the present invention so as to provide for their independent benefits.

However, by using preferred combinations of ingredients (e.g., repair agents, protection agents, moisturizing agents and/or repair agents), the ingredients in the present invention are able to work together synergistically so as to achieve unexpected and improved benefits with respect to skin cleansing, skin moisturizing, skin repair and skin protection. For example, in a preferred embodiment, the present invention provides for a gentle cleansing of the skin, while also improving overall skin tone, texture and clarity, while further brightening, protecting and repairing the skin, a combination of beneficial results not before provided by products on the marketplace or disclosed in the art. In working synergistically, ingredients are able to maintain the function of each ingredient and/or solution when all are combined together. To this end, the ingredients are able to cleanse, moisturize, repair and protect, without eliminating or cancelling the independent function or effects of any one ingredient. The inventors have now discovered after trials and experiments that the ingredients described herein, when combined together, need not be altered with respect to their independent functions when not combined.

The inventors have conducted the following study. Trials were conducted of 12 random participants ranging from ages 30-79. A 12-week trial was conducted using wipes of the present invention. Participants were instructed to use wipes generously morning and night, and not to rinse off. Participants were also instructed not to use any other product on the face except the wipes of the present invention. After 12 weeks, there was visible considerable repairing to the skin. The skin was visibly improved with respect to reduced puffiness under the eyes, dark circles under the eyes were reduced, pore size tightened and became smaller, spots faded, and the participants overall appeared rejuvenated, bright, clear of blemishes and youthful.

In one exemplary embodiment of the present invention, the invention may be used to clean the skin in order to prevent infection that may result from medical procedures such as surgery and/or injection. In another exemplary embodiment, the present invention may be used to clean the skin in order to prevent infection that may result from every day and more traumatic skin injuries. In a preferred embodiment, the invention may include chlothexidine glucoside to assist in preventing infection resulting from surgery, injection and/or skin injuries.

The present invention may be illustrated by way of the following non-limiting examples.

### Example 1

An exemplary embodiment of the invention comprises a woven or non-woven fabric, wherein the fabric incorporates a solution or composition that comprises glycerin, steareth-20, chrysin, N-hydroxysuccinimide, palmitoyl oligopeptide-1 and palmitoyl tetrapeptide-7, propanediol, iris florentina root extract, zinc and retinyl palmitate, and chrysin.

### Example 2

An exemplary embodiment of the invention comprises a woven or non-woven fabric, wherein the fabric incorporates a solution or composition that comprises steareth-20, chrysin, N-hydroxysuccinimide, palmitoyl oligopeptide-1 and palmitoyl tetrapeptide-7, propanediol, iris florentina root extract, zinc and retinyl palmitate, chrysin, glycolic acid and phytic acid.

### Example 3

An exemplary embodiment of the invention comprises a cosmetic or medical device comprising a woven or non-woven fabric, wherein the fabric incorporates a solution or composition that comprises glycerin, steareth-20, chrysin, N-hydroxysuccinimide, palmitoyl oligopeptide-1 and palmitoyl tetrapeptide-7, propanediol, iris florentina root extract, zinc and retinyl palmitate, chrysin, at least one cleansing agent (e.g., glycolic acid and/or chlorhexidine glucoside), at least one moisturizing agent (e.g., glycerin), and at least one protection agent (e.g., aloe vera and/or apple).

### Example 4

An exemplary embodiment of the invention comprises a cosmetic or medical device comprising a woven or non-woven fabric, wherein the fabric incorporates a solution or composition that comprises glycerin, steareth-20, chrysin, N-hydroxysuccinimide, palmitoyl oligopeptide-1 and palmitoyl tetrapeptide-7, propanediol, iris florentina root extract, zinc and retinyl palmitate, chrysin, chlorhexidine glucoside, glycerin, sodium PCA, aloe vera and apple.

### Example 5

An exemplary embodiment of the invention comprises a cosmetic or medical device comprising a woven or non-woven fabric, wherein the fabric incorporates a solution or composition that comprises water, sodium citrate, gluconolactone, pentylene glycol, lavandula angustifolia (lavender) oil, pyrus malus (apple) fruit extract, aloe barbadensis leaf extract, avena sativa (oat) kernel extract, chamomilla recututa (matricaria) flower extract, cucumis sativus (cucumber) fruit extract, althaea officinalis root extract, fraxinus excelsior bark extract, glycerin, sodium PCA, butylene glycol, niacinamide, silanetriol, caprylyl/capryl glucoside, sodium benzoate, steareth-20, tetrasodium EDTA, N-hydroxysuccinimide, chrysin, palmitoyl tripeptide-1, palmitoyl tetrapeptide-7, lactic acid and phytic acid.

While aspects of the present invention are described herein and illustrated in the accompanying drawings, those of ordinary skill in the art will appreciate that the invention, in its broadest aspects, has further applicability.

It will be appreciated that the above description is merely exemplary in nature and is not intended to limit the present disclosure, its application or uses. Therefore, it is intended that the present disclosure not be limited to the particular examples illustrated by the drawings and described in the specification as the best mode presently contemplated for carrying out the teachings of the present disclosure, but that the scope of the present disclosure will include any embodiments falling within the foregoing description and the appended claims.

We claim:

1. A cosmetic or medical device comprising: a woven or non-woven fabric, the fabric incorporating a solution or composition that comprises:
a first repair agent that comprises glycerin, steareth-20, chrysin, N-hydroxysuccinimide, palmitoyl oligopeptide-1 and palmitoyl tetrapeptide-7,
a second repair agent that comprises propanediol, iris florentina root extract, zinc and retinyl palmitate,
chrysin.

2. The cosmetic or medical device of claim 1, wherein the first repair agent is haloxyl.

3. The cosmetic or medical device of claim 1, wherein the second repair agent is cytobiol.

4. The cosmetic or medical device of claim 1, further comprising at least one compound selected from palmitoyl tripeptide-1 and palmitoyl tetrapeptide-7.

5. The cosmetic or medical device of claim 1, further comprising a cleansing agent.

6. The cosmetic or medical device of claim 1, further comprising at least one cleansing agent selected from glycolic acid, lactic acid, steareth-20, caprylyl, capryl, glucoside and chlorhexidine glucoside.

7. The cosmetic or medical device of claim 1, further comprising a third repair agent.

8. The cosmetic or medical device of claim 1, further comprising at least one third repair agent selected from vitamin B3, chamomile, oat, cucumber, marshmallow root, bark, bark extract, tetrapeptide-7, glycolic acid and phytic acid.

9. The cosmetic or medical device of claim 8, wherein the bark or bark extract is fraxinus excelsior bark extract.

10. The cosmetic or medical device of claim 1, further comprising a moisturizing agent.

11. The cosmetic or medical device of claim 10, wherein the moisturizing agent is glycerin.

12. The cosmetic or medical device of claim 1, further comprising at least one protection agent.

13. The cosmetic or medical device of claim 12, wherein the protection agent is selected from aloe vera and apple.

14. The cosmetic or medical device of claim 1, wherein the woven or non-woven fabric is a facial wipe or a body wipe.

15. A cosmetic or medical device comprising: a woven or non-woven fabric, the fabric incorporating a solution or composition that comprises:
a first repair agent that comprises glycerin, steareth-20, chrysin, N-hydroxysuccinimide, palmitoyl oligopeptide-1 and palmitoyl tetrapeptide-7,
a second repair agent that comprises propanediol, iris florentina root extract, zinc and retinal palmitate,
chrysin,
at least one cleansing agent,
at least one moisturizing agent,
at least one protection agent.

16. The cosmetic or medical device of claim 15, wherein the first repair agent is haloxyl.

17. The cosmetic or medical device of claim 15, wherein the second repair agent is cytobiol.

18. The cosmetic or medical device of claim 15, wherein the at least one cleansing agent is selected from the group consisting of glycolic acid, lactic acid, steareth-20, caprylyl, capryl, glucoside and chlorhexidine glucoside.

19. The cosmetic or medical device of claim 15, wherein the at least one moisturizing agent is selected from glycerin and sodium PCA.

20. The cosmetic or medical device of claim 15, wherein the at least one protecting agent is selected from the group consisting of aloe vera and apple.

21. The cosmetic or medical device of claim 15, further comprising at least one compound selected from palmitoyl tripeptide-1 and palmitoyl tetrapeptide-7.

22. The cosmetic or medical device of claim 15, further comprising at least one third repair agent selected from vitamin B3, chamomile, oat, cucumber, marshmallow root, bark, bark extract, tetrapeptide-7, glycolic acid and phytic acid.

23. The cosmetic or medical device of claim 15, wherein the woven or non-woven fabric is a facial wipe or a body wipe.
